(19) United States
(12) Patent Application Publication
(10) Pub. No.: US 2002/0137767 A1
(43) Pub. Date: Sep. 26, 2002

(54) Methods for treating addictive disorders

(76) Inventors: David John Heal, Nottinghamshire (GB); Patricia Lesley Needham, Nottinghamshire (GB)

Correspondence Address: LAHIVE & COCKFIELD, 28 STATE STREET, BOSTON, MA, 02109, US

(21) Appl. No.: 10/096,186

(22) Filed: Mar. 11, 2002

Related U.S. Application Data
(63) Continuation of application No. 09/896,510, filed on Jun. 28, 2001, now abandoned.
Application No. 09/896,510 is a continuation of application No. 09/350,943, filed on Jul. 9, 1999, now abandoned.

(51) Int. Cl. A61K 31/47

(52) U.S. Cl. 514/307

(57) ABSTRACT

The present invention provides a method of reducing cravings to food or an addictive substance in a mammal comprising administering an effective amount of a D₁/D₅ antagonist or a D₁/D₅ partial agonist to a mammal in need thereof. These compounds are also useful in the treatment of addictive drug induced psychoses. Suitably the addictive substance is cocaine, amphetamine, nicotine, opiates, tobacco or alcohol. The addictive substance may be ecstasy. The preferred compound is (+)-1-[1-(2-chlorophenyl)cyclopropyl]-7-hydroxy-6-methoxy-2-methyl-1,2,3,4-tetrahydroisoquinoline and salts thereof.

### BACKGROUND OF THE INVENTION

The problems associated with cravings to food or addictive substances are legion. The societal consequences of obesity, tobacco and drug addiction and abuse are well known. Indeed, substantial investments in both time and money have been made to combat both the causes and effects of substance addiction and abuse. As part of this research, many efforts have been directed to preventing relapse by subjects, either during or after a particular treatment program. For example, as reported by Berger et al., “Haloperidol antagonism of cue-elicited cocaine craving,” Lancet, 1996 February 24;347(9000):504-8, haloperidol has been tested as a compound for reducing desire for an abused substance. However, haloperidol is also reported to have significant adverse side affects. Therefore, it would be desirable to provide compositions and methods to reduce a subject's cravings for food or an addictive substance.

### SUMMARY OF THE INVENTION

The present invention relates to (+)-1-[1-(2-chlorophenyl)cyclopropyl]-7-hydroxy-6-methoxy-2-methyl-1,2,3,4-tetrahydroisoquinoline and pharmaceutically acceptable salts thereof and their use in the treatment of drug misuse or other addictive disorders.

Tetrahydroisoquinoline compounds of formula I

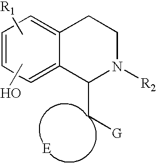

and pharmaceutically acceptable salts thereof, in which:

R₁ represents one or more substituents selected from H, halo, hydroxy, alkyl of 1 to 3 carbon atoms (optionally substituted by hydroxy), alkoxy of 1 to 3 carbon atoms, alkylthio of 1 to 3 carbon atoms, alkylsulphinyl of 1 to 3 carbon atoms, alkylsulphonyl of 1 to 3 carbon atoms, nitro, cyano, polyhaloalkyl of 1 to 3 carbon atoms, polyhaloalkoxy of 1 to 3 carbon atoms, phenyl (optionally substituted by one or more substituents selected from halo, alkyl of 1 to 3 carbon atoms or alkoxy of 1 to 3 carbon atoms), or R₁ is carbamoyl optionally alkylated by one or two alkyl groups each independently of 1 to 3 carbon atoms;

R₂ represents an aliphatic group containing 1 to 3 carbon atoms optionally substituted by hydroxy or alkoxy containing 1 to 3 carbon atoms;

E represents an alkylene chain containing 2 to 5 carbon atoms optionally substituted by one or more alkyl groups containing 1 to 3 carbon atoms,

and G represents phenyl or phenyl substituted by one or more substituents which may be the same or different, and which are independently alkyl of 1 to 3 carbon atoms, alkoxy of 1 to 3 carbon atoms, halo, hydroxy, polyhaloalkyl of 1 to 3 carbon atoms, polyhaloalkoxy of 1 to 3 carbon atoms, cyano, alkylthio of 1 to 3 carbon atoms, alkylsulphinyl of 1 to 3 carbon atoms, alkylsulphonyl of 1 to 3 carbon atoms, phenyl (optionally substituted by one or more substituents selected from halo, alkyl of 1 to 3 carbon atoms or alkoxy of 1 to 3 carbon atoms), carbamoyl optionally alkylated by one or two alkyl groups each independently of 1 to 3 carbon atoms, or G represents a phenyl ring having fused thereto a heterocyclic or aromatic carbocyclic ring;

and O-acylated derivatives thereof are disclosed in WO93/13073, which is incorporated herein by reference, as being useful for achieving analgesia and in the treatment of psychoses, Parkinson's disease, Lesch-Nyan syndrome, attention deficit disorder or cognitive impairment or in the relief of drug dependence or tardive dyskinesia.

### DETAILED DESCRIPTION OF THE INVENTION

One of the compounds disclosed in WO93/13073 (BTS 73 947) (+)-1-[1-(2-chlorophenyl)cyclopropyl]-7-hydroxy-6-methoxy-2-methyl-1,2,3,4-tetrahydroisoquinoline was reported to be in clinical development as a anti-psychotic. In J. Psychopharmacol 1996, Vol. 10, Part 2, Suppl. Page A6, Needham PL et al., and Eur. Neuropsycho-pharmacol 1996 Vol.6, Suppl. 3, page 105, Needham PL et al., it is disclosed that in behavioral models, predictive of antipsychotic and side-effect potential (using male CD rats), BTS 73 947 potently inhibited locomotion (ED₅₀=0.7 mg/kg ip) and stereotypy (ED₅₀=2.4 mg/kg ip), induced by d-amphetamine sulphate (2.5 mg/kg sc and 10 mg/kg sc, respectively), demonstrating ˜3.5-fold greater potency against limbic DA function. Its peak effect was 4 h post-dose, with >6 h duration of action. By comparison. BTS 73 947 was only weakly cataleptic (ED₅₀=25.4 mg/kg ip) giving an excellent separation between the ED₅₀ dose to inhibit amphetamine locomotion (predictive of antipsychotic activity) and to induce catalepsy (predictive of extrapyrarnidal side-effects (EPS): ratio=36. The behavioral tests predict that BTS 73 947 will have an atypical profile with potent, long lasting antipsychotic activity and a minimal propensity to produce EPS. In addition it was reported that BTS 73 947 selectively protected the D₁-like receptors of male CD rats (Charles River) from EEDQ (1-ethoxycarbonyl-2-ethoxy-1,2-dihydroquinoline) inactivation (ED₅₀=1 mg/kg po), with no effect on D₂-like or 5-HT₂ receptors. Functional antagonism of D₁-receptors in vivo was demonstrated by the induction of D₂-mediated atypical jerking in male SD rats (Olac), pretreated with BTS 73 947: ED₁₅ (dose induced 15 jerks in the 15 min test period)=3.4 mg/kg po. These results demonstrate that BTS 73 947 is a selective, high affinity D₁/D₅ antagonist. The hydrobromide salt of BTS 73 947 is BTS 72 428.

The present invention provides a method of reducing cravings to food or an addictive substance in a mammal comprising administering an effective amount of a D₁/D₅ antagonist or a D₁/D₅ partial agonist to a mammal in need thereof. Preferably the D₁/D₅ antagonist of D₁/D₅ partial agonist is administered at a daily dosage range of about 0.01 to about 500 mg/kg. The present invention also provides methods of reducing cravings to food or an addictive substance in a mammal comprising administering to a mammal in need thereof an effective amount of a D₁/D₅ antagonist or a D₁/D₅ partial agonist, in combination with one or more anti-obesity compounds, serotonin receptor agonists and antagonists, antipsychotics, anxiolytics, antidepressants, dopaminergic agonists, anticonvulsants, mood stimulants, cocaine-like agonists, cocaine catalytic antibodies, and/or alcohol and opiate antagonist drugs.

Suitably the addictive substance is cocaine, amphetamine, nicotine, opiates, tobacco or alcohol. The addictive substance may also be metamphetamine, MDMA (ecstasy), a cannabinoid, LSD, MDA, MDE or PCP. The term opiates includes heroin and morphine.

The preferred compound is (+)-1-[1-(2-chlorophenyl)cyclopropyl]-7-hydroxy-6-methoxy-2-methyl-1,2,3,4-tetrahydroisoquinoline and salts thereof.

In a preferred aspect the present invention provides a method of treating dependence on an addictive substance comprising administering (+)-1-[1-(2-chlorophenyl)cyclopropyl]-7-hydroxy-6-methoxy-2-methyl-1,2,3,4-tetrahydroisoquinoline to achieve beneficial effects which are maintained despite increased self-administration of the addictive substance.

In another aspect the present invention provides a method of treating drug abuse comprising administering to a mammal in need thereof a dose of (+)-1-[1-(2-chlorophenyl)cyclopropyl]-7-hydroxy-6-methoxy-2-methyl-1,2,3,4-tetrahydroisoquinoline which is sufficient to maintain its effect despite increased self administration of the abuse substance. Suitable doses are given below.

The present invention also provides a method of reducing cravings to food or an addictive substance comprising administering a therapeutically effective amount of (+)-1-[1-(2-chlorophenyl)cyclopropyl]-7-hydroxy-6-methoxy-2-methyl-1,2,3,4-tetrahydroisoquinoline or a pharmaceutically acceptable salt thereof, to a mammal, particularly a human being, in need thereof.

Conditions which may be advantageously treated with the compounds of the present invention include disorders arising from drug misuse including drug withdrawal symptoms, aiding in the cessation of smoking, aiding in the prevention of relapse after cessation of drug use and similar use in the treatment of other addictive disorders such as compulsive gambling, compulsive shopping disorder and compulsive sexual disorder.

In another aspect the present invention provides a method of treating addictive-drug-induced psychoses comprising administering a therapeutically effective amount of (+)-1-[1-(2-chlorophenyl)cyclopropyl]-7-hydroxy-6-methoxy-2-methyl-1,2,3,4-tetrahydroisoquinoline or a salt thereof to a mammal, particularly a human being, in need thereof.

Addictive drugs which may cause psychoses include benzodiazepines, cannabinoids, LSD, MDMA, MDA, PCP, opiates including heroin and morphine, amphetamine, cocaine and alcohol.

In yet another aspect the present invention provides methods of treating dependence on an addictive substance comprising administering to a mammal in need thereof, a dose of (+)-1-[1-(2-chlorophenyl)cyclopropyl]-7-hydroxy-6-methoxy-2-methyl-1,2,3,4-tetrahydroisoquinoline in combination with one or more anti-obesity compounds, serotonin receptor agonists and antagonists, antipsychotics, anxiolytics, antidepressants, dopaminergic agonists, anticonvulsants, mood stimulants, cocaine-like agonists, cocaine catalytic antibodies, and/or alcohol and opiate antagonist drugs.

The present invention also includes pharmaceutical compositions comprising a therapeutically effective amount of (+)-1-[1-(2-chlorophenyl)cyclopropyl]-7-hydroxy-6-methoxy-2-methyl-1,2,3,4-tetrahydroisoquinoline or a salt thereof together with a pharmaceutically acceptable diluent or carrier which are useful in reducing cravings to food or an addictive substance.

In another aspect the present invention comprises the use of (+)-1-[1-(2-chlorophenyl)cyclopropyl]-7-hydroxy-6-methoxy-2-methyl-1,2,3,4-tetrahydroisoquinoline in the manufacture of a medicament for treating cocaine abuse.

As used hereinafter, the term “active compound” denotes (+)-1-[1-(2-chlorophenyl)cyclopropyl]-7-hydroxy-6-methoxy-2-methyl-1,2,3,4-tetrahydroisoquinoline or a salt thereof. In therapeutic use, the active compound may be administered orally, rectally, parenterally or topically, preferably orally. Thus the therapeutic compositions of the present invention may take the form of any of the known pharmaceutical compositions for oral, rectal, parenteral or topical administration. Pharmaceutically acceptable carriers suitable for use in such compositions are well known in the art of pharmacy. The compositions of the invention may contain 0.1-99% by weight of active compound. The compositions of the invention are generally prepared in unit dosage form. Preferably the unit dosage of active ingredient is 1-500 mg. The excipients used in the preparation of these compositions are the excipients known in the pharmacist's art.

Compositions for oral administration are the preferred compositions of the invention and these are the known pharmaceutical forms for such administration, for example tablets, capsules, syrups and aqueous or oil suspensions. The excipients used in the preparation of these compositions are the excipients known in the pharmacist's art. Tablets may be prepared by mixing the active compound with an inert diluent such as calcium phosphate in the presence of disintegrating agents, for example maize starch, and lubricating agents, for example magnesium stearate, and tableting the mixture by known methods. The tablets may be formulated in a manner known to those skilled in the art so as to give a sustained release of the compounds of the present invention. Such tablets may, if desired, be provided with enteric coatings by known methods, for example by the use of cellulose acetate phthalate. Similarly, capsules, for example hard or soft gelatin capsules, containing the active compound with or without added excipients, may be prepared by conventional means and, if desired, provided with enteric coatings in a known manner. The tablets and capsules may conveniently each contain 1 to 500 mg of the active compound. Other compositions for oral administration include, for example, aqueous suspensions containing the active compound in an aqueous medium in the presence of a non-toxic suspending agent such as sodium carboxymethylcellulose, and oily suspensions containing a compound of the present invention in a suitable vegetable oil for example arachis oil.

Solid oral dosage forms may be formulated in a manner known to those skilled in the art so as to give a sustained release of the active compound. Enteric coated, solid oral dosage forms comprising compositions of the present invention may be advantageous, depending on the nature of the active compound. Various materials, for example shellac and/or sugar, may be present as coatings, or to otherwise modify the physical form of the oral dosage form. For example tablets or pills may, if desired, be provided with enteric coatings by known methods, for example by the use of cellulose acetate phthalate and/or hydroxy propyl methylcellulose phthalate.

Capsules and/or caplets (for example hard or soft gelatin capsules) comprising the active compound (with or without added excipients such as a fatty oil), may be prepared by conventional means and, if desired, provided with enteric coatings in a known manner. The contents of the capsule and/or caplet may be formulated using known methods to give sustained release of the active compound.

Liquid oral dosage forms comprising compositions of the present invention may be an elixir, suspension and/or syrup (for example, aqueous suspensions containing the active compound in an aqueous medium in the presence of a non-toxic suspending agent [such as sodium carboxymethylcellulose] and/or oily suspensions containing the active compound in a suitable vegetable oil [such as arachis oil and/or sunflower oil]). Liquid oral dosage forms may also comprise one or more sweetening agent, flavoring agent, preservatives and/or mixtures thereof.

The active compound may be formulated into granules with or without additional excipients. The granules may be ingested directly by the patient or they may be added to a suitable liquid carrier (for example water) before ingestion. The granules may contain disintegrants (for example a pharmaceutically acceptable effervescent couple formed from an acid and a carbonate or bicarbonate salt) to facilitate dispersion in the liquid medium.

Preferably each of the above oral dosage forms may contain from about 1 mg to about 1000 mg, more preferably from about 5 mg to about 500 mg (for example 10 mg, 25 mg, 50 mg, 100 mg, 200 mg, or 400 mg) of the active compound.

Compositions of the invention suitable for rectal administration are the known pharmaceutical forms for such administration, for example, suppositories with hard fat, semi-synthetic glyceride, cocoa butter and/or polyethylene glycol bases.

Pharmaceutical compositions may also be administered parenterally (for example subcutaneously, intramuscularly, intradermally and/or intravenously [such as by injection and/or infusion] in the known pharmaceutical dosage forms for parenteral administration (for example sterile suspensions in aqueous and/or oily media and/or sterile solutions in suitable solvents, preferably isotonic with the blood of the intended patient). Parenteral dosage forms may be sterilised (for example by micro-filtration and/or using suitable sterilising agents [such as ethylene oxide]). Optionally one or more of the following pharmaceutically acceptable adjuvants suitable for parenteral administration may be added to parenteral dosage forms: local anaesthethetics, preservatives, buffering agents and/or mixtures thereof. Parenteral dosage forms may be stored in suitable sterile sealed containers (for example ampoules and/or vials) until use. To enhance stability during storage the parenteral dosage form may be frozen after filling the container and fluid (for example water) may be removed under reduced pressure.

Pharmaceutical compositions may be administered nasally in known pharmaceutical forms for such administration (for example sprays, aerosols, nebulised solutions and/or powders). Metered dose systems known to those skilled in the art (for example aerosols and/or inhalers) may be used.

Pharmaceutical compositions may be administered to the buccal cavity (for example sub-lingually) in known pharmaceutical forms for such administration (for example slow dissolving tablets, chewing gums, troches, lozenges, pastilles, gels, pastes, mouthwashes, rinses and/or powders).

Compositions for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as a mineral oil, petrolatum and/or a wax, for example paraffin wax or beeswax, together with a potential transdermal accelerant such as dimethyl sulphoxide or propylene glycol. Alternatively the active compounds may be dispersed in a pharmaceutically acceptable cream or ointment base. The amount of active compound contained in a topical formulation should be such that a therapeutically effective amount of the compound is delivered during the period of time for which the topical formulation is intended to be on the skin.

The compounds of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as a suspension or solution in a pharmaceutically acceptable oil of the compound to be infused for example in the form of a very sparingly water-soluble derivative such as a dodecanoate salt or (b) solid in the form of an implanted support, for example of a synthetic resin or waxy material, for the compound to be infused. The support may be a single body containing all the compound or a series of several bodies each containing part of the compound to be delivered. The amount of active compound present in an internal source should be such that a therapeutically effective amount of the compound is delivered over a long period of time.

In some formulations it may be beneficial to use the compounds of the present invention in the form of particles of very small size, for example as obtained by fluid energy milling.

In the compositions of the present invention the active compound may, if desired, be associated with other compatible pharmacologically active ingredients.

This invention is further illustrated by the following examples which should not be construed as limiting. The contents of all references and published patents and patent applications cited throughout the application are hereby incorporated by reference.

### EXAMPLE 1

The use of compounds of the present invention in the manufacture of pharmaceutical compositions is illustrated by the following Example. In this Example, the term “active compound” denotes any compound of the invention but particularly any compound which is the final product of the Example.

a) Capsules

In the preparation of capsules, 10 parts by weight of active compound and 240 parts by weight of lactose are de-aggregated and blended. The mixture is filled into hard gelatin capsules, each capsule containing a unit dose or part of a unit dose of active compound.

b) Tablets

Tablets are prepared from the following ingredients.

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch are de-aggregated, blended and the resulting mixture is granulated with a solution of the polyvinylpyrrolidone in ethanol. The dry granulate-is blended with the magnesium stearate and the rest of the starch. The mixture is then compressed in a tabletting machine to give tablets each containing a unit dose or a part of a unit dose of active compound.

c) Enteric Coated Tablets

Tablets are prepared by the method described in (b) above. The tablets are enteric coated in a conventional manner using a solution of 20% cellulose acetate phthalate and 3% diethyl phthalate in ethanol:dichloromethane (1:1).

d) Suppositories

In the preparation of suppositories, 100 parts by weight of active compound is incorporated in 1300 parts by weight of triglyceride suppository base and the mixture formed into suppositories each containing a therapeutically effective amount of active ingredient.

In the following examples, the test compound was (+)-1-[1-(2-chlorophenyl)cyclopropyl]-7-hydroxy-6-methoxy-2-methyl-1,2,3,4tetrahydroisoquinoline hydrobromide.

### EXAMPLE 2

Subjects: The subjects were four male rhesus monkeys (Macaca mulatta), weighing 5.7-8.1 kg and maintained on a diet of 3-4 monkey biscuits and one piece of fresh fruit per day. During the week, all food was delivered after the experimental session, whereas at weekends, food was delivered between 9 a.m. and noon. Water was freely available at all times. The monkeys were housed in a humidity and temperature controlled room with a 12 h light-dark cycle (lights on from 7 a.m. to 7 p.m.).

Apparatus: Each monkey was housed individually in a well-ventilated, stainless steel chamber (56×71×69 cm)which included an operant panel (28×28 cm) mounted on the front wall. Three response keys were arranged in a horizontal row 3.2 cm from the top of the operant panel. Each key could be transilluminated by red or green stimulus lights (Superbright LEDs). An externally mounted pellet dispenser delivered 1 g fruit-flavored food pellets to a food receptacle beneath the operant response panel. A computer, located in a separate room, controlled the operant panels and data collection.

Discrimination Training: Discrimination training was conducted 5 days per week during daily sessions composed of multiple cycles. Each cycle consisted of a 15 min time-out period followed by a 5 min response period. During the time-out, all stimulus lights were off, and responding had no scheduled consequences. During the response period, the right and left response keys were transilluminated red or green, and monkeys could earn up to 10 food pellets by responding under a FR 30 schedule of food presentation. For one monkey, the left key was illuminated green and the right key was illuminated red; the colors of the response-keys were reversed for the other three monkeys. The center key was not illuminated at any time and responding on it had no scheduled consequences. If all available food pellets were delivered before the end of the 5 min response period, the stimulus lights were turned off and responding had no scheduled consequences for the remainder of the 5 min period.

On training days, monkeys were given either saline or 0.40 mg/kg cocaine, i.m., 10 min before the response period. Following the administration of saline, responding on only the green key (the saline-appropriate key) produced food, whereas following administration of 0.40 mg/kg cocaine, only responding on the red key (the drug-appropriate key) produced food. Responses on the inappropriate key reset the FR requirement on the appropriate key. Sessions consisted of 1 to 5 cycles and, if cocaine was administered, this occurred only during the last cycle. Thus, training days consisted of 0 to 5 saline cycles followed by 0 or 1 cocaine cycle.

During each response period, 3 dependent variables were determined:

1) Percent injection-appropriate responding prior to delivery of the first reinforcer.

2) Percent injection-appropriate responding for the entire response period.

3) Response Rate

Monkeys meeting the following criteria during the training day immediately proceeding the test day and in at least 6 of 7 consecutive training sessions before this, were used for discrimination testing:

1) the percent injection-appropriate responding prior to delivery of the first reinforcer was ≧80% for all cycles;

2) the percent injection-appropriate responding for the entire cycle was ≧90% for all cycles;

3) Response rates during saline training cycles were >0.5 responses per second.

If responding did not meet criterion levels of discrimination performance, then training was continued until criterion levels of performance were obtained for at least two consecutive days.

Discrimination Testing: Test sessions were identical to training sessions except that responding on either key produced food, and the test compound was administered using a Pretreatment Protocol. In this protocol, a cumulative dose-effect curve for cocaine (0.013-1.3 mg/kg) was determined either alone or following pretreatment with the test compound, which was administered 20 min before the first dose of cocaine.

Mean data from saline and drug cycles during the training day immediately proceeding the initial test day served as the control data for the subsequent test day.

Data Analysis: The Percent Cocaine-Appropriate Responding and the Response Rate were plotted as a function of the dose of cocaine (log scale). Where possible, the ED₅₀ value for cocaine was determined by drawing a line between the points above and below 50% cocaine-appropriate responding, and then using linear regression to interpolate the dose that would produce 50% cocaine-appropriate responding. ED₅₀ values for cocaine administered alone and following pretreatment with the test compound were then compared.

Drugs: Cocaine hydrochloride was dissolved in sterile saline. The test compound was dissolved in 1% lactic acid in distilled water.

Results

Control mean saline-appropriate responding=99.8% (±0.2) and 100% appropriate responding was obtained during cocaine cycles. Mean response rates were 2.19 (±0.20) and 2.61 (±0.22) responses/sec during saline and drug training cycles, respectively.

ED₅₀ values for cocaine are shown in Table 1. Administration of cocaine alone produced a dose-dependent increase in cocaine-appropriate responding in all four monkeys. Complete substitution was obtained at the training dose of cocaine (0.4 mg/kg) in all monkeys, and a higher dose of 1.3 mg/kg usually decreased response rates. Pretreatment with 0.01 mg/kg of the test compound produced a rightward shift in the cocaine dose-effect curve and a 3-fold increase in the cocaine ED₅₀ value in monkey 2, but it had no effect on the cocaine discrimination dose-effect curve in the other three monkeys. A higher dose of 0.032 mg/kg of the test compound produced rightward shifts in the cocaine dose-effect curves in all four monkeys. The test compound (0.01 and 0.032 mg/kg) also eliminated responding during the first one to three cycles of the cumulative cocaine dose-effect curve determination (i.e. in combination with 0.013 and 0.04 mg/kg cocaine). However, monkeys responded after administration of higher cocaine doses, thereby permitting evaluation of the effects on cocaine discrimination. Interestingly, response rates following administration of the highest dose of cocaine (1.3 mg/kg) were often higher following test compound pretreatment than for cocaine alone, suggesting that the test compound attenuated the rate-decreasing effects of high cocaine doses.

TABLE 1
Comparison of ED₅₀ values (mg/kg) for cocaine administered either
alone or after pretreatment with test compound

| | Dose Test Compound (mg/kg) | | |
|---|---|---|---|
| Monkey | Cocaine Alone | 0.01 | 0.032 |
| 1 | 0.23 | 0.23 | 0.72 |
| 2 | 0.23 | 0.72 | 0.72 |
| 3 | 0.23 | 0.23 | 0.071 |
| 4 | 0.23 | 0.24 | a |

^{a}Could not be determined because all points were greater than 50% cocaine-appropriate responding.

These findings suggest that the test compound antagonizes the discriminative stimulus effects and possibly also the rate decreasing effects of cocaine at doses that also produce effects on response rates.

### EXAMPLE 3

Subjects: The subjects were four male rhesus monkeys (Macaca mulatta). Each monkey was maintained on a diet of 3 monkey biscuits and one piece of fresh fruit per day in addition to fruit-flavored pellets delivered during operant sessions (see below). Water was freely available at all times. The monkeys were housed in a humidity and temperature controlled room with a 12 hr light-dark cycle (lights on from 7 a.m. to 7 p.m.).

Monkeys were surgically implanted with double-lumen silicone rubber catheters (inside diameter 0.7 mm, outside diameter 2.0 mm) to facilitate concurrent delivery of cocaine and treatment compounds. Catheters were implanted in the jugular or femoral vein and exteriorised in the midscapular region. All surgical procedures were performed under aseptic conditions. Monkeys were sedated with ketamine (5 mg/kg, s.c.), and anesthesia was induced with sodium thiopental (10 mg/kg, i.v). Monkeys received 0.05 mg/kg atropine, to reduce salivation. Following insertion of a tracheal tube, anesthesia was maintained with isoflurane (1-1.5% in oxygen). After surgery, monkeys were administered aspirin or acetaminophen (80-160 mg/day; p.o.) for 3 days and Procaine Penicillin 0 (300,000 units/day, i.m.) every day for 5 days. The i.v. catheter was protected by a tether system consisting of a custom-fitted nylon vest connected to a flexible stainless steel cable and fluid swivel (Lomir Biomedical; Malone, N.Y), which permitted the monkeys to move freely. Catheter patency was periodically evaluated by i.v. administration of the short-acting barbiturate methohexital (3 mg/kg i.v.) or ketamine (2-3 mg/kg i.v.). The catheter was considered patent if i.v. administration of methohexital or ketamine produced loss of muscle tone within 10 seconds after its administration.

Apparatus: Each monkey was housed individually in a well-ventilated stainless steel chamber (64×64×79 cm) which included an operant panel (28×28 cm) mounted on the front wall. Three response keys (6.4×6.4 cm) were arranged in a horizontal row 3.2 cm from the top of the operant panel. Each key could be transilluminated by red or green stimulus lights (Superbright LEDs). An externally-mounted pellet dispenser delivered 1 g fruit-flavored food pellets to a food receptacle beneath the operant response panel. Two syringe pumps were mounted above each cage for delivery of saline or drug solutions through the intravenous catheters. Operant panels and data collection were controlled by a computer through a MED-PC interface.

Training: Food and i.v. drug or saline injections were available during three alternating components: a 5 min food component, a 100-min drug component, and a second 5 min food component. Both food and i.v. injections were available under a FR 30 schedule of reinforcement. During the two food components, the response key was transilluminated red. During the drug component, the response key was transilluminated green. Following the delivery of each food pellet or drug injection, there was a 10 sec timeout period, during which the stimulus light illuminating the center response key was turned off and responding had no scheduled consequences. The food and drug components were separated by 5-min timeout periods when the response key was dark, and responding had no scheduled consequences. The entire food/drug/food session lasted 120 min.

In addition to the food/drug/food session described above, monkeys were also given the opportunity to self-administer additional food pellets during supplementary food sessions. During these sessions, food was available under a FR30/Timeout 10 sec schedule, and a maximum of 25 pellets per session could be earned. These food sessions provided additional enrichment opportunities for the monkeys and behavioral information relevant for the evaluation of prolonged treatment drug effects.

During training, the solution available for self-administration during the drug component was alternated between 0.032 mg/kg/inj cocaine (the maintenance dose of cocaine) and saline. Each period of cocaine or saline availability usually lasted from 3 to 10 days. Monkeys were trained until they met the following criteria for stable cocaine self-administration: 1) three consecutive days during which the response rate during the drug component of each session differed by no more than 20% from the mean drug component response rate and there was no upward or downward trend; and 2) rapid saline extinction as indicated by a decrease in drug component response rates on the first day of saline substitution.

Evaluation of Test Compound: The effects of the test compound (0.0032-0.10 mg/kg) on cocaine self-administration and food-maintained behavior were evaluated using the standard pretreatment test procedure. In this procedure, the test compound was administered i.m. 20-min prior to a test session during which a test unit dose of cocaine was available during the drug component. Two series of studies are described here. In the first, the unit dose of cocaine was 0.0032 mg/kg/inj (at or near the peak of each monkey's cocaine self-administration dose-effect curve) and the effects of pretreatment with each dose of test compound were determined in single sessions for all monkeys. In the second series of studies, the effects of pretreatment with each of two doses of the test compound (0.003 and 0.01 mg/kg) on the entire cocaine dose-effect function were determined. In these studies, the dose of cocaine was systematically varied for single test sessions after pretreatment with each dose of the test compound. Both the dose of cocaine and the pretreatment dose of the test compound were varied across test sessions in an irregular order among monkeys.

At the conclusion of each pretreatment test in either series of studies, training conditions (availability of saline or the maintenance dose of cocaine) were reinstated. Test sessions generally were conducted on Tuesdays and Fridays, and either saline or the maintenance dose of cocaine was available during training sessions for the remainder of the week. On occasion, another dose of cocaine was substituted for the maintenance dose to insure that the position of the cocaine dose-effect function in individual monkeys was stable. In addition, test days were occasionally omitted to allow several days of saline substitution.

Data Analysis: The dependent variables were the response rates during each food and drug component. The response rate was calculated as [total # responses (component duration—S timeouts)]. Control response rates for each food and drug component during availability of each unit dose of cocaine were defined as the response rate obtained when that unit dose of cocaine was available and no pretreatment was administered. The ED₅₀ value for the test compound during each food or drug component was defined as the dose of the test compound that decreased rates of cocaine or food self-administration to 50% of control response rates. The ED₅₀ values were determined where possible by linear regression from the linear portion of the test compound dose-effect curve.

For subsequent studies, in which the unit dose of cocaine was varied and the pretreatment dose of the test compound was held constant, response rates are graphed as a function of the unit dose of cocaine. Control cocaine dose-effect curves determined in the absence of pretreatment are visually compared to cocaine dose-effect curves determined following pretreatment with the test compound.

Drugs: Cocaine hydrochloride was dissolved in saline. A stock solution of 10 mg/ml of the test compound was prepared using a vehicle of 1% lactic acid in distilled water, and dilutions were made with distilled water. Aseptic precautions were taken in every phase of cocaine solution preparation and dispensing. Cocaine solutions were filter-sterilised using a 0.22 micron Millipore Filter and stored in sterile, pyrogen-free vials. Sterility of the entire fluid path for drug solutions was maintained throughout the study. Each unit dose of cocaine was delivered i.v. in an injection volume of 0.1 ml. Doses of the test compound were delivered i.m. in a volume of 0.2-3.0 ml.

Results

Table 2 shows control rates of cocaine and food-maintained responding during availability of 0.003 mg/kg/inj of cocaine for all 4 monkeys. Table 3 shows the effects of the test compound on rates of cocaine- and food-maintained responding in individual monkeys when the unit dose of cocaine during the component of drug availability was 0.003 mg/kg/inj.

TABLE 2
Control rates of responding (responses sec) maintained by cocaine
and food during availability of 0.003 mg/kg/ml cocaine.

| | Component | | |
|---|---|---|---|
| Monkey | Drug | Food #1 | Food #2 |
| 1 | 0.36 | 4.23 | 4.30 |
| 2 | 0.36 | 6.25 | 6.11 |
| 3 | 0.32 | 3.23 | 3.43 |
| 4 | 1.08 | 4.44 | 2.77 |

TABLE 3
ED₅₀ values (mg/kg) for the test compound in decreasing response rates
maintained by cocaine and food when cocaine self-administration was
maintained by (0.003 mg/kg/inj. The first food component (Food #1)
preceded the drug component and the second food component
(Food #2) followed the drug component.

| | Component | | |
|---|---|---|---|
| Monkey | Drug | Food #1 | Food #2 |
| 1 | 0.002 | 0.003 | 0.011 |
| 2 | 0.001 | 0.002 | 0.006 |
| 3 | 0.004 | 0.002 | 0.002 |
| 4 | a | a | a |

a = ED₅₀ value could not be determined

These experiments indicate that treatment with the test compound diminishes cocaine self-administration behavior. On average, even the lower dose of the test compound decreased responding maintained by lower doses of cocaine and shifted the peak of the dose-effect function for cocaine self-administration 3-fold to the right. Importantly, the effects of the treatment were not surmounted by increased self-administration of higher unit doses of cocaine by any monkey. The effects of the low pretreatment dose of the test compound (0.003 mg/kg) on food-maintained behavior were generally variable but moderate across monkeys. A higher dose of the test compound (0.01 mg/kg) appreciably decreased food-maintained response rates in the first component of food availability. These effects generally dissipated in the second food component and, on average, response rates returned nearly to control values. Interestingly, recovery of food-maintained responding was not observed following sessions in which a relatively ineffective dose of cocaine (0.00032 mg/kg) was available for self-administration. Thus the restoration of control rates of food-maintained performance after cocaine self-administration may reflect the mutually antagonistic effects of self-administered cocaine and the pretreatment drug of the test compound.

Overall the test compound shifted the dose-effect function for cocaine self-administration rightward and downward, i.e. the effects of pretreatment were not fully overcome by increasing the unit dose of cocaine available for self-administration. This represents a significant advantage for the test compound in the treatment of cocaine addiction.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of reducing cravings to food or an addictive substance in a mammal comprising administering an effective amount of a D₁/D₅ antagonist or a D₁/D₅ partial agonist to a mammal in need thereof.

2. The method of claim 1 wherein the D₁/D₅ antagonist of D₁/D₅ partial agonist is administered at a daily dosage range of about 0.01 to about 500 mg/kg.

3. The method of either claim 1 or claim 2 wherein the addictive substance is cocaine, amphetamine, nicotine, opiates, tobacco or alcohol.

4. The method of claim 1 in which the compound is (+)-1-[1-(2-chlorophenyl)cyclopropyl]-7-hydroxy-6-methoxy-2-methyl-1,2,3,4-tetrahydroisoquinoline.

5. A method of treating dependence on an addictive substance comprising administering a dose of (+)-1-[1-(2-chlorophenyl)cyclopropyl]-7-hydroxy-6-methoxy-2-methyl-1,2,3,4-tetrahydroisoquinoline which is sufficient to achieve beneficial effects which are maintained despite increased self administration of the addictive substance.

6. The method of claim 5 wherein the addictive substance is cocaine, amphetamine, nicotine, opiates, tobacco or alcohol.

7. A method of reducing food cravings in a mammal comprising administering (+)-1-[1-(2-chlorophenyl)cyclopropyl]-7-hydroxy-6-methoxy-2-methyl-1,2,3,4-tetrahydroisoquinoline to a mammal in need thereof.

8. A method of treating drug addiction comprising administering to a mammal in need thereof a dose of (+)-1-[1-(2-chlorophenyl)cyclopropyl]-7-hydroxy-6-methoxy-2-methyl-1,2,3,4-tetrahydroisoquinoline which is sufficient to maintain its effect despite increased self-administration of the addictive drug.

9. The method of claim 8 in which the dose is selected from 10 mg, 25 mg, 50 mg, 100 mg, 200 mg, or 400 mg.

10. The method of claim 9 in which the addictive drug is cocaine.

11. A method of treating addictive-drug-induced psychoses comprising administering a therapeutically effective amount of (+)-1-[1-(2-chlorophenyl)cyclopropyl]-7-hydroxy-6-methoxy-2-methyl-1,2,3,4-tetrahydroisoquinoline or a salt thereof to a mammal, particularly a human being, in need thereof.

12. The method of claim 11 in which the addictive drug is selected from one or more of the following: a benzodiazepine; a cannabinoid, LSD, MDMA, MDA, PCP, an opiate including heroin and morphine, amphetamine, cocaine and alcohol.

13. A method of reducing cravings to food or an addictive substance in a mammal comprising administering to a mammal in need thereof an effective amount of a D₁/D₅ antagonist or a D₁/D₅ partial agonist in combination with a compound selected from anti-obesity compounds, serotonin receptor agonists, serotonin receptor antagonists, antipsychotics, anxiolytics, antidepressants, dopaminergic agonists, anticonvulsants, mood stimulants, cocaine-like agonists, cocaine catalytic antibodies, alcohol antagonist drugs and opiate antagonist drugs.

14. A method of treating dependence on an addictive substance comprising administering to a mammal in need thereof, a dose of (+)-1-[1-(2chlorophenyl)cyclopropyl]-7-hydroxy-6-methoxy-2-methyl-1,2,3,4-tetrahydroisoquinoline in combination with a compound selected from anti-obesity compounds, serotonin receptor agonists, serotonin receptor antagonists, antipsychotics, anxiolytics antidepressants, dopaminergic agonists, anticonvulsants, mood stimulants, cocaine-like agonists, cocaine catalytic antibodies, alcohol antagonist drugs and opiate antagonist drugs.
